(19) United States
(12) Patent Application Publication
(10) Pub. No.: US 2008/0279967 A1
(43) Pub. Date: Nov. 13, 2008

(54) Composition and method for increasing the metabolism of free fatty acids and facilitating a favorable blood lipid

(76) Inventors: Marvin HEUER, Mississauga (CA); Ken CLEMENT, Mississauga (CA); Shan CHAUDHURI, Mississauga (CA); Michele MOLINO, Mississauga (CA); Phil APONG, Mississauga (CA)

Correspondence Address: TORYS LLP, 79 WELLINGTON STREET WEST, SUITE 3000, BOX 270, TD CENTRE, TORONTO, ON, M5K 1N2, CA

(21) Appl. No.: 11/747,208

(22) Filed: May 10, 2007

(51) Int. Cl. A61K 36/00; A61P 3/04

(52) U.S. Cl. 424/725

(57) ABSTRACT

A dietary supplement comprising at least an effective amount of Cissus quadrangularis extract and an effective amount of γ-butyrobetaine. The ingredients of the present dietary supplement act substantially to facilitate a more favorable blood lipid profile, and increase the catabolism of free fatty acids via the phosphorylation of perilipins. In additional aspects of the present invention γ-butrybetaine ethyl ester is added to the dietary supplement to provide further synergistic or additional benefits. Both a composition and a method are provided by the present disclosure.

### RELATED APPLICATIONS

This application is related to co-pending U.S. patent application Ser. No. ______, entitled “Composition for inducing lipolysis and increasing the metabolism of free fatty acids via increased nitric oxide levels” filed on May 10, 2007, the contents of which are hereby incorporated by reference in there entirety.

### FIELD OF THE INVENTION

The present invention relates to a dietary supplement for facilitating a more favorable blood lipid profile and increasing the metabolism of free fatty acids via increased nitric oxide levels. More specifically, the present invention relates to a dietary supplement comprising a combination of Cissus quadrangularis extract and γ-butyrobetaine.

### BACKGROUND OF THE INVENTION

Obesity, a condition of excessive body fat, generally results from more food being consumed than is being used. Stemming from excessive body fat, several health-related concerns have been linked to obesity and being overweight, such as increased morbidity, hypertension, coronary heart disease, type 2 diabetes mellitus, stroke and even some forms of cancer (Curioni C, Andre C, Veras R. Weight reduction for primary prevention of stroke in adults with overweight or obesity. Cochrane Database Syst Rev. Oct. 18, 2006; (4):CD006062). Obesity has become an increasingly widespread and predominant health concern. According to the World Health Organization (WHO) obesity is considered a multifactorial chronic disease which is increasing in frequency (Curioni C, Andre C, Veras R. Weight reduction for primary prevention of stroke in adults with overweight or obesity. Cochrane Database Syst Rev. Oct. 18, 2006; (4):CD006062).

One of the main contributing factors in obesity is overeating, which results in an excess of energy being consumed in relation to the amount of energy being expended by an individual. This excess energy is then commonly stored as fat. A simplified determination of an individual's body weight is essentially governed by the net effect of energy consumed versus energy expended. Daily energy expenditure consists of three components: basal metabolic rate, adaptive thermogenesis and physical activity (Westerterp KR. Diet induced thermogenesis. Nutr Metab (Lond). Aug. 18, 2004; 1(1):5). All of the aforementioned components must be in a balance with energy expenditure in an individual, that is, energy or food intake being such that an individual does not gain nor lose body weight. Therefore, in order for a person to lose body weight from a reduction in adipose tissue, more energy must be expended by the individual than is taken into the body.

With the unprecedented rise in obesity throughout the world, there exists both a need and want from individuals for improved aids, methods and interventions directed to reducing body fat and maintaining lowered levels of body fat. These needs have led to intensive study of the various mechanisms of fat metabolism. One such mechanism that has shown promise is the arginine-nitric oxide pathway. Nitric oxide (NO), which is synthesized from arginine by all cell types, has been shown to be a key signal molecule involved in adipose tissue biology by influencing adipogenesis and insulin-stimulated glucose uptake.

There are four major ways in which NO has been shown to influence energy metabolism. Firstly, NO has been shown to increase the phosphorylation of hormone-sensitive lipase and perilipin. Perilipin is a protein that coats lipid droplets in adipocytes and acts to protect triglycerides from hormone-sensitive lipases, which break lipids into glycerol and free fatty acids. When the perilipin is phosphorylated it changes conformation and exposes stored lipids to hormone-sensitive lipase-mediated lipolysis, hence stimulation of lipolysis.

Second, NO has been shown to stimulate the phosphorylation of adenosine-3′,5′-monophoshate activated protein kinase. Activation of these kinases causes a decrease in levels of malonyl-CoA, which plays a key role in chain elongation in fatty acid biosynthesis by providing 2-carbon units to fatty acids thus comitting them to fatty acid chain synthesis. Additionally, activated kinases decrease the expression of genes related to lipogenesis and gluconeogenesis. Thirdly, NO has been shown to increase blood flow to insulin-sensitive tissue, thus promoting substrate uptake and product removal. Lastly, NO has been shown to activate the expression of peroxisome proliferator-activated receptors leading to enhanced mitochondrial biogenesis and oxidative phosphorylation.

### SUMMARY OF THE INVENTION

The present invention is directed towards a dietary supplement, comprising at least an effectivea therapeutically effective amount of Cissus quadrangularis extract, and an effectivea therapeutically effective amount of γ-butyrobetaine. The ingredients of the present dietary supplement act substantially simultaneously to facilitate a more favorable blood lipid profile, and increase the catabolism of free fatty acids via the phosphorylation of perilipins. Both a composition and a method are provided by the present disclosure.

### DETAILED DESCRIPTION OF THE INVENTION

In the following description, for the purposes of explanations, numerous specific details are set forth in order to provide a thorough understanding of the present invention. It will be apparent, however, to one of ordinary skill in the art that the present invention may be practiced without these specific details.

The present invention is directed towards a dietary supplement, for facilitating a more favorable blood lipid profile and increasing the catabolism of free fatty acids via the phosphorylation of perilipins, comprising a combination of Cissus quadrangularis extract and γ-butyrobetaine. An aspect of the present invention comprises at least an extract of Cissus quadrangularis and γ-butyrobetaine.

The term ‘γ-butyrobetaine’ as used herein is understood to represent gamma-butyrobetaine, also known as, butyrobetaine, deoxycarnitine, actinine, 4-butyrobetaine, or 4-trimethylamniobutyrate. Additionally, as used herein, ‘γ-butyrobetaine’ also includes derivatives of gamma-butyrobetaine such as esters, amides, and salts, as well as other derivatives, including derivatives having pharmacoproperties upon metabolism to an active form.

The term ‘more favorable blood lipid profile’ as used herein is understood to represent and be characterized or influenced by any one or more of the following: a reduction in low-density lipoprotein (LDL), a reduction in cholesterol, a lowering of fasting blood glucose levels, and increased levels of high-density lipoproteins (HDL).

Cissus quadrangularis Extract

Cissus quadrangularis is a plant indigenous to India where it is part of traditional medicine. Extracts of Cissus quadrangularis contain sterols, vitamin C, and tannins with antimicrobial in addition to antioxidant activity (Chidambara Murthy K N, Vanitha A, Mahadeva Swamy M, Ravishankar G A. Antioxidant and antimicrobial activity of Cissus quadrangularis L. J Med Food, 2003. 6(2): p. 99-105). With respect to Cissus quadrangularis as a weight reduction agent, clinical studies have shown that a group taking an extract of Cissus quadrangularis for 6-weeks lost more weight, had lower cholesterol, LDL and fasting blood glucose levels. The experimental group also displayed increased HDL levels as compared to a placebo group (Oben J E, Mandob D, Fomekong G, Momo C. The effect of an extract of Cissus quadrangularis (Cylaris™) on weight and serum lipids in obese patients in Cameroon: a randomized double-blind clinical trial. Presented at Paris Anti-Obesity Therapies. May 2006; Oben J E, Enyegue D M, Fomekong G I, Soukontoua Y B, Agbor G A. The effect of Cissus quadrangularis (CQR-300) and a Cissus formulation (CORE) on obesity and obesity-induced oxidative stress. Lipids Health Dis. Feb. 4, 2007;6:4). The lowering levels of LDL and raising levels of HDL has been associated with improved health, particularly in conjunction with weight reduction (Dattilo A M, Kris-Etherton P M. Effects of weight reduction on blood lipids and lipoproteins: a meta-analysis. Am J Clin Nutr, 1992. 56(2): p. 320-8).

It is herein understood by the inventors that lowering levels of LDL and cholesterol, as well as increasing levels of HDL will lead to a more favorable blood lipid profile, facilitating improved health via weight reduction.

An embodiment of the present invention comprises Cissus quadrangularis extract. A serving of the nutritional supplement contains from about 0.05 g to about 0.50 g of Cissus quadrangularis extract.

γ-butyrobetaine (GBB) and Derivatives

γ-butyrobetaine is an intermediate in carnitine biosynthesis in mammals. It is synthesized, from trimethyl lysine, in almost all cell types and then excreted into the blood to be reabsorbed by the kidney and liver. After reabsorption, GBB is converted to carnitine by γ-butyrobetaine dioxygenase. This conversion to carnitine is extremely efficient and so the presence of γ-butyrobetaine in urine is very small (Vaz F M, Wanders R J A. Carnitine biosynthesis in mammals. Biochem J. 2002;361:417-429).

Since γ-butyrobetaine is a precursor to carnitine, its administration has been studied as a way to increase carnitine levels in the body. Carnitine acts as a carrier molecule for fatty acids across the inner mitochondrial membrane. In order for free fatty acids to be catabolized, they must first enter the mitochondria of a cell such that β-oxidation may take place to produce energy. Fatty acids are first activated with the addition of coenzyme A (CoA), then bound to carnitine and transferred across the mitochondrial inner membrane, after which the carnitine is removed.

As an additional effect, administration of γ-butyrobetaine to rats (Sjakste N, Kleschyov J L, Baumane L, Dzintare M, Meirena D, Sjakste J, Sydow K, Munzel T, Kalvinsh I. Endothelium- and nitric oxide-dependent vasorelaxing activities of gamma-butyrobetaine esters: possible link to the antiischemic acitivites of mildronate. Eur J Pharmacol. Jul. 8, 2004; 495(1):67-73 (Abstract)), showed elevated vasodilating activities. These vasodilating activities were attributed to increases in nitric oxide concentrations in blood. Since nitric oxide has been shown to increase energy metabolism, it is herein understood by the inventors that increases in NO levels will lead to increased catabolism of fatty acids, which will increase the utilization of adipocytes leading to reduced fat content in the body, by mechanisms that were previously discussed.

An embodiment of the present invention comprises γ-butyrobetaine or derivatives thereof. A serving of the nutritional supplement contains from about 0.005 g to about 0.050 g of γ-butyrobetaine or derivatives thereof.

In an embodiment of the present invention, which is set forth in greater detail in Example 1, the dietary supplement comprises an extract of green tea, γ-butyrobetaine, and γ-butyrobetaine ethyl ester. The dietary supplement is provided in any acceptable and suitable oral dosage form as known in the art to induce lipolysis and increase the metabolism of free fatty acids via increased nitric oxide levels.

While, not wishing to be bound by theory, the present invention is comprised of components that have been shown to reduce levels of LDL, cholesterol, and fasting blood glucose as well as increase levels of HDL. The facilitation of a more favorable blood lipid profile will result in improved health via weight reduction (Dattilo A M, Kris-Etherton P M. Effects of weight reduction on blood lipids and lipoproteins: a meta-analysis. Am J Clin Nutr, 1992. 56(2): p. 320-8).

Additionally, the present invention comprises components that have been shown to lead to, via increased nitric oxide levels, increased phosphorylation of hormone-sensitive lipase and perilipin. It is herein understood by the inventors that increased phosphorylation of perilipin will result in greater likelihood of lipids being broken down by lipases, leading to increased release of free fatty acids.

Furthermore, increased nitric oxide levels will yield greater phosphorylation of adenosine-3′,5′-monophosphate activated protein kinase, which will cause a decrease in levels of malonyl-CoA and decrease the expression of genes related to lipogenesis and gluconeogenesis. It is herein understood by the inventors that decreased malonyl-CoA will result in reduced chain elongation of fatty acids and thus a decrease in fatty acid biosynthesis.

In addition, the present invention comprises components that have been shown to lead to, via increased nitric oxide levels, elevated expression of peroxisome proliferator-activated receptors. It is herein understood by the inventors that increased expression of peroxisome proliferator-activated receptors will enhance mitochondrial biogenesis and oxidative phosphorylation, resulting in elevated metabolism of free fatty acids.

Further to the aforementioned functions, the present invention comprises components that have been shown to lead to, via increased nitric oxide levels, elevated blood flow to insulin-sensitive tissue. It is herein understood by the inventors that increased blood flow will promote substrate uptake and product removal, thus increasing the movement of free fatty acids throughout the body.

Additional embodiments of the present invention may also include portions of the composition as fine-milled ingredients. U.S. Non-Provisional patent application Ser. No. 11/709,526 entitled “Method for Increasing the Rate and Consistency of Bioavailability of Supplemental Dietary Ingredients” filed Feb. 21, 2007, which is herein fully incorporated by reference, discloses a method of increasing the rate of bioavailability following oral administration of components comprising supplemental dietary compositions by the process of particle-milling.

Furthermore, additional embodiments of the present invention may be incorporated into specific controlled-release solid dosage forms. U.S. Non-Provisional patent application Ser. No. 11/709,525 entitled “Method for a Supplemental Dietary Composition Having a Multi-Phase Dissolution Profile” filed Feb, 21, 2007, which is herein fully incorporated by reference, discloses a method of achieving a solid oral dosage form with multiple dissolution characteristics for the release of active ingredients.

According to various embodiments of the present invention, the dietary supplement may be consumed in any form. For instance, the dosage form of the dietary supplement may be provided as, e.g., a powder beverage mix, a liquid beverage, a ready-to-eat bar or drink product, a capsule, a liquid capsule, a tablet, a caplet, or as a dietary gel. The preferred dosage forms of the present invention are as a caplet or as a liquid capsule.

Furthermore, the dosage form of the dietary supplement may be provided in accordance with customary processing techniques for herbal and dietary supplements in any of the forms mentioned above. Additionally, the dietary supplement set forth in the example embodiment herein disclosed may contain any appropriate number and type of excipients, as is well known in the art. By way of ingestion of the composition of the present invention, a method for substantially simultaneously facilitating a more favorable blood lipid profile and increasing the catabolism of free fatty acids via via the phosphorylation of perilipins. The method of the present invention comprises at least the step of administering to an individual an effective amount of the composition of the present invention. Although the following example illustrates the practice of the present invention in one of its embodiments, the example should not be construed as limiting the scope of the invention. Other embodiments will be apparent to one of skill in the art from consideration of the specifications and example.

### EXAMPLES

### Example 1

A dietary supplement comprising the following ingredients per serving is prepared for consumption as a caplet:

About 0.15 g of Cissus quadrangularis extract which is standardized for 2.5% phytosterols, about 0.01 g of γ-butyrobetaine (GBB), and about 0.025 g of γ-butryrobetaine ethyl ester.

### Example 2

A dietary supplement comprising the following ingredients per serving is prepared for consumption as a caplet:

About 0.15 g of Cissus quadrangularis extract which is standardized for 2.5% phytosterols, about 0.01 g of γ-butyrobetaine (GBB), about 0.025 g of γ-butyrobetaine ethyl ester, and about 0.01 g of theobromine.

### Example 3

A dietary supplement comprising the following ingredients per serving is prepared for consumption as a caplet:

About 0.15 g of Cissus quadrangularis extract which is standardized for 2.5% phytosterols, about 0.01 g of γ-butyrobetaine (GBB), about 0.025 g of γ-butyrobetaine ethyl ester, and about 0.46 g of green tea extract green tea extract which is standarized for 90% polyphenols, 75% catechins, 45% epigallocatechin gallate.

Extensions and Alternatives

In the foregoing specification, the invention has been described with a specific embodiment thereof; however, it will be evident that various modifications and changes may be made thereto without departing from the broader spirit and scope of the invention.

What is claimed:

1. A composition comprising an effective amount of Cissus quadrangularis extract, and an effective amount of γ-butyrobetaine;
wherein the ingredients act substantially simultaneously to facilitate a more favorable blood lipid profile, and increase catabolism of free fatty acids via the phosphorylation of perilipins.

2. The composition of claim 1 wherein the presence of malonyl-CoA is decreased, thereby reducing fatty acid biosynthesis.

3. The composition of claim 1 wherein expression of peroxisome proliferators-activated activated receptors is increased, thereby increasing fatty acid metabolism by enhancing mitochondrial biogenesis and oxidative phosphorylation.

4. The composition of claim 1 wherein blood flow to insulin-sensitive tissue is enhanced, thereby promoting movement of free fatty acids throughout the body.

5. A method for weight reduction in a mammal comprising the step of administering to a mammal a composition comprising Cissus quadrangularis extract and γ-butyrobetaine or derivatives of γ-butyrobetaine wherein the composition causes perilipin phosphorylation and substantially simultaneously maintains a favorable lipoprotein profile.

6. The method of claim 5 wherein the presence of malonyl-CoA is increased, thereby reducing fatty acid biosynthesis.

7. The method of claim 5 wherein expression of peroxisome proliferators-activated receptors is increased, thereby increasing fatty acid metabolism by enhancing mitochondrial biogenesis and oxidative phosphorylation.

8. The method of claim 5 wherein blood flow to insulin-sensitive tissue is enhanced, thereby promoting movement of free fatty acids throughout the body.

9. A composition comprising from about 0.05 g to about 0.50 g of Cissus quadrangularis extract, and from about 0.005 g to about 0.050 g of γ-butyrobetaine;
wherein the Cissus quadrangularis extract and the γ-butyrobetaine act substantially simultaneously to facilitate a more favorable blood lipid profile, and increase catabolism of free fatty acids via the phosphorylation of perilipins.

10. The composition of claim 9 wherein the amount of Cissus quadrangularis extract is about 0.150 g and the amount of γ-butyrobetaine is about 0.010 g.

11. The composition of claim 9 wherein at least a portion of one or more ingredients is fine-milled.

12. The composition of claim 9 wherein the Cissus quadrangularis extract and γ-butyrobetaine are part of a solid oral dosage form having a multi-phasic rate of dissolution.

13. The composition of claim 12 wherein said multi-phasic rate of dissolution comprises a first-phase and a second-phase; whereby said first-phase has a first rate of dissolution said second-phase has a second rate of dissolution.

14. The composition of claim 13, further comprising a third-phase, whereby said third-phase has a third rate of dissolution.
